(19) United States
(12) Patent Application Publication
(10) Pub. No.: US 2018/0251821 A1
(43) Pub. Date: Sep. 6, 2018

(54) DETECTION METHOD AND DETECTION KIT FOR NUCLEIC ACID MOLECULES

(71) Applicant: CHANG GUNG UNIVERSITY, Taoyuan City (TW)

(72) Inventors: Chiuan-Chian CHIOU, TAOYUAN CITY (TW); Tai-Long CHEN, TAOYUAN CITY (TW); Ji-Dung LUO, TAOYUAN CITY (TW)

(21) Appl. No.: 15/660,226

(22) Filed: Jul. 26, 2017

(30) Foreign Application Priority Data
Mar. 3, 2017 (TW) 106107094

(51) Int. Cl. C12Q 1/68

(52) U.S. Cl. CPC C12Q 1/6818; C12Q 2600/158; C12Q 1/6883

(57) ABSTRACT

A method for detecting a target nucleic acid fragment includes the following steps: preparing a nucleic acid probe and an intercalating dye configured to bind to double-stranded nucleic acids, wherein the nucleic acid probe includes a nucleic acid chain, a fluorescent reporter, and a fluorescent quencher, the fluorescent reporter is conjugated to a first end of the nucleic acid chain, and the fluorescent quencher is conjugated to a second end of the nucleic acid chain opposing to the first end; binding the nucleic acid probe to the target nucleic acid fragment to form a partial double stranded structure; intercalating the intercalating dye in the partial double stranded structure, so that, after the intercalating dye is excited by an irradiation, the fluorescent reporter is excited through the intercalating dye to emit a fluorescence signal; and detecting the target nucleic acid fragment according to the fluorescence signal.

### CROSS REFERENCE TO RELATED APPLICATIONS

This Non-provisional application claims priority under 35 U.S.C. § 119(a) on Patent Application No(s). 106107094 filed in Taiwan, Republic of China on Mar. 3, 2017, the entire contents of which are hereby incorporated by reference.

### BACKGROUND OF THE INVENTION

### Field of Invention

The invention relates to a detection method and a detection kit. In particular, the invention relates to a method and a kit for detecting a target nucleic acid fragment.

### Related Art

Oligonucleotides refer to short chain deoxyribonucleic acid (DNA) molecules or ribonucleic acid (RNA) molecules, which are widely used in gene detection, scientific research and forensic identification. In the field of molecular biology, oligonucleotides are often made into single-stranded molecules with specific nucleic acid sequences for gene synthesis, polymerase chain reaction, deoxyribonucleic acid sequencing and gene pool construction, and used as molecular probes.

The nucleic acid sequences of an oligonucleotide probe can be used to detect deoxyribonucleic acid molecules or ribonucleic acid molecules (target nucleic acids) with specific nucleic acid sequences according to the users' requirement, depending on the matching relationship between nucleotides. Currently, it is known that there is a fluorescent probe that is an oligonucleotide probe coupled with fluorescent reporters at one end. When the fluorescent probe binds to a complementary nucleic acid sequence, providing excitation light with specific wavelength can make fluorescent reporters undergo fluorescence resonance energy transfer (FRET) with another fluorescent molecule intercalated in a double stranded structure (i.e., an intercalating dye which is configured to bind to double-stranded nucleic acids), such that the fluorescent reporters produce emission light with specific wavelength.

However, the fluorescent probe would form intramolecular or intermolecular partial double-stranded structures, producing a significant background signal, which further affects the interpretation of the amplification curve or the melting curve. Although a proper probe sequence design may slightly reduce the background signal, most of the gene detection must target specific nucleic acid sites, making the probe sequence design not be modified flexibly. Therefore, the background signal is difficult to be eliminated. The defect makes the technology of the fluorescent probe lack of practicality, and difficult to be widely accepted.

### SUMMARY OF THE INVENTION

An aspect of the disclosure is to provide a method and a kit for detecting a target nucleic acid fragment, which can effectively eliminate the background signal generated by a probe itself, and can be used in a polymerase chain reaction to efficiently quantify a target nucleic acid or to perform genotyping.

A method for detecting a target nucleic acid fragment is provided. The method comprises the following steps: preparing a nucleic acid probe and an intercalating dye which is configured to bind to double-stranded nucleic acids, wherein the nucleic acid probe includes a nucleic acid chain, a fluorescent reporter, and a fluorescent quencher, the fluorescent reporter is conjugated to a first end of the nucleic acid chain, and the fluorescent quencher is conjugated to a second end of the nucleic acid chain opposing to the first end; binding the nucleic acid probe to the target nucleic acid fragment to form a partial double stranded structure; intercalating the intercalating dye in the partial double stranded structure, so that, after the intercalating dye is excited by an irradiation, the fluorescent reporter is excited through the intercalating dye to emit a fluorescence signal; and detecting the target nucleic acid fragment according to the fluorescence signal.

In one embodiment, when the nucleic acid probe is not bound to the target nucleic acid fragment, the fluorescent quencher absorbs the fluorescence signal emitted by the fluorescent reporter.

In one embodiment, the nucleic acid chain is a peptide nucleic acid chain, a locked nucleic acid chain, a ribonucleic acid chain, or a deoxyribonucleic acid chain.

In one embodiment, the nucleic acid chain has a length ranging from 15 mers to 70 mers.

In one embodiment, after the fluorescent quencher absorbs the fluorescence signal emitted by the fluorescent reporter, the fluorescent quencher does not emit emission light or emits emission light with a wavelength greater than that of the fluorescence signal of the fluorescent reporter.

In one embodiment, the fluorescent reporter is selected from the group consisting of: HEX, Cy5, ROX, Bodipy 630/650, and LCRed 640.

In one embodiment, the fluorescent quencher is selected from the group consisting of: DABCYL, BHQ, Iowa Black, QSY, and carboxytetramethyl rhodamine.

In one embodiment, the intercalating dye is selected from the group consisting of: SYBR Green I, SYBR Gold, ethidium bromide, LC Green, and EvaGre en.

A kit for detecting a target nucleic acid fragment is also provided. The kit comprises an intercalating dye which is configured to bind to double-stranded nucleic acids, and a nucleic acid probe. The nucleic acid probe includes a nucleic acid chain, a fluorescent reporter, and a fluorescent quencher. The fluorescent reporter is conjugated to a first end of the nucleic acid chain. The fluorescent quencher is conjugated to a second end of the nucleic acid chain opposing to the first end. The intercalating dye intercalates in a partial double stranded structure formed after the nucleic acid probe binds to the target nucleic acid fragment, and the target nucleic acid fragment is detected according to a fluorescence signal emitted by the fluorescent reporter which is excited through the intercalating dye.

In one embodiment, when the nucleic acid probe is not bound to the target nucleic acid fragment, the fluorescent quencher absorbs the fluorescence signal emitted by the fluorescent reporter.

In one embodiment, the nucleic acid chain is a peptide nucleic acid chain, a locked nucleic acid chain, a ribonucleic acid chain, or a deoxyribonucleic acid chain.

In one embodiment, the nucleic acid chain has a length ranging from 15 mers to 70 mers.

In one embodiment, after the fluorescent quencher absorbs the fluorescence signal emitted by the fluorescent reporter, the fluorescent quencher does not emit emission light or emits emission light with a wavelength greater than that of the fluorescence signal of the fluorescent reporter.

In one embodiment, the fluorescent reporter is selected from the group consisting of: HEX, Cy5, ROX, Bodipy 630/650, and LCRed 640.

In one embodiment, the fluorescent quencher is selected from the group consisting of: DABCYL, BHQ, Iowa Black, QSY, and carboxytetramethyl rhodamine.

In one embodiment, the intercalating dye is selected from the group consisting of: SYBR Green I, SYBR Gold, ethidium bromide, LC Green, and EvaGreen.

As mentioned above, the method and the kit of the present disclosure are used to detect target nucleic acid fragments using the nucleic acid probe including the fluorescent reporter and the fluorescent quencher, and the intercalating dye. When the nucleic acid probe forms intramolecular or intermolecular partial double-stranded structures (i.e., a non-specific match occurs), the fluorescent quencher absorbs the fluorescence signal with specific wavelength emitted by the fluorescent reporter due to the fluorescent reporter being close to the fluorescent quencher. That is, when the nucleic acid probe is not bound to the target nucleic acid fragment, the fluorescent quencher absorbs the fluorescence signal emitted by the fluorescent reporter. Therefore, when using in the polymerase chain reaction, the background signal generated by the non-specific match can be effectively eliminated, so that the amplification curve or the melting curve is clearer and easier to interpret to efficiently quantify the target nucleic acid fragments or to perform genotyping.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a flow chart of a method for detecting a target nucleic acid fragment according to an embodiment of the present disclosure.

FIG. 2A is a schematic diagram of fluorescence signals generated by a method for detecting a target nucleic acid fragment according to an embodiment of the present disclosure.

FIGS. 2B and 2C are schematic diagrams of nucleic acid probes forming a non-specific match.

FIG. 3A is a schematic diagram of a melting curve showing that nucleic acid probes are perfectly matched or mismatched with target nucleic acid fragments.

FIG. 3B is a schematic diagram of a melting curve obtained by differentiating the melting curve of FIG. 3A.

FIG. 4 is an amplification curve obtained using a nucleic acid probe that does not have a fluorescent quencher.

FIG. 5 is an amplification curve obtained using a nucleic acid probe having a fluorescent quencher.

FIG. 6 is a melting curve obtained using a nucleic acid probe that does not have a fluorescent quencher in Experimental example 1 of the present disclosure.

FIG. 7 is a melting curve obtained using a nucleic acid probe having a fluorescent quencher in Experimental example 1 of the present disclosure.

FIGS. 8A, 8B and 8C are melting curves obtained using different fluorescent reporters and fluorescent quenchers in Experimental example 2 of the present disclosure.

FIGS. 9A and 9B are melting curves obtained in Experimental example 3 of the present disclosure.

FIGS. 10A and 10B are schematic diagrams showing the formation of a non-specific match of nucleic acid probes that do not have fluorescent quenchers.

### DETAILED DESCRIPTION OF THE INVENTION

The embodiments and experimental examples of the present invention will be apparent from the following detailed description, which proceeds with reference to the accompanying drawings, wherein the same references relate to the same elements.

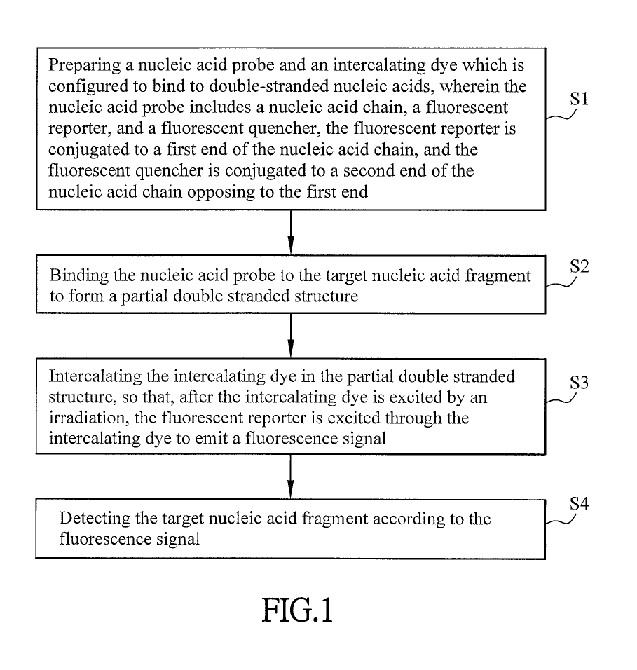

FIG. 1 is a flow chart of a method for detecting a target nucleic acid fragment according to an embodiment of the present disclosure. As shown in FIG. 1, the method comprises the following steps: preparing a nucleic acid probe and an intercalating dye which is configured to bind to double-stranded nucleic acids, wherein the nucleic acid probe includes a nucleic acid chain, a fluorescent reporter, and a fluorescent quencher, the fluorescent reporter is conjugated to a first end of the nucleic acid chain, and the fluorescent quencher is conjugated to a second end of the nucleic acid chain opposing to the first end (step S1); binding the nucleic acid probe to the target nucleic acid fragment to form a partial double stranded structure (step S2); intercalating the intercalating dye in the partial double stranded structure, so that, after the intercalating dye is excited by an irradiation, the fluorescent reporter is excited through the intercalating dye to emit a fluorescence signal (step S3); and detecting the target nucleic acid fragment according to the fluorescence signal (step S4).

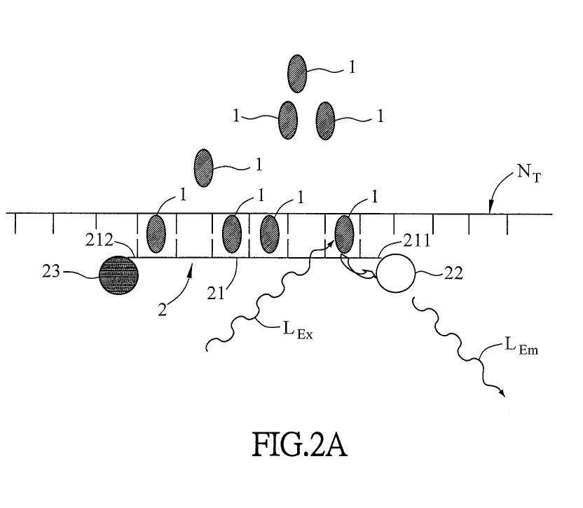

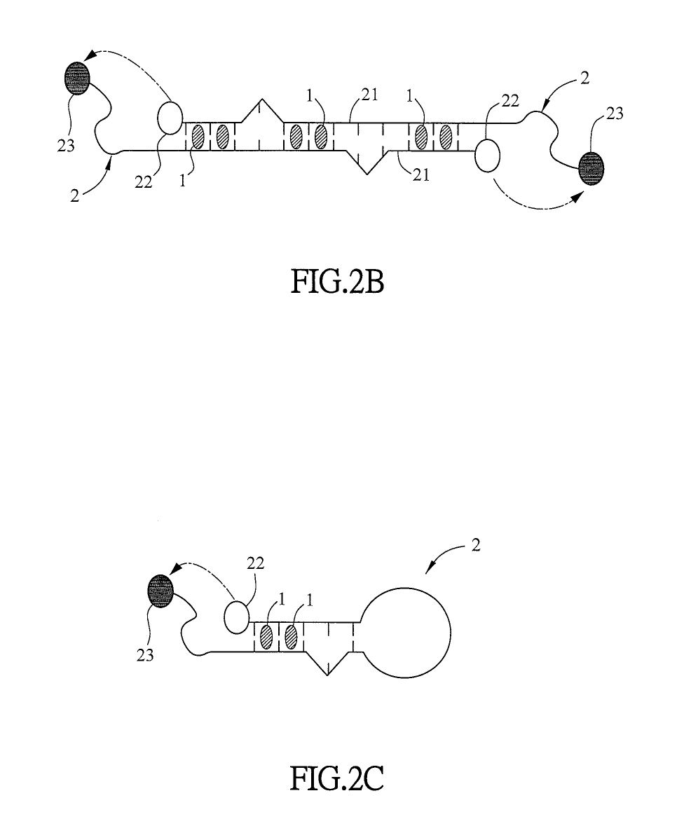

FIG. 2A is a schematic diagram of fluorescence signals generated by a method for detecting a target nucleic acid fragment according to an embodiment of the present disclosure. Referring to FIG. 1 and FIG. 2A, in step S1, an intercalating dye 1 which is configured to bind to double-stranded nucleic acids and a nucleic acid probe 2 are prepared. The intercalating dye which is configured to bind to double-stranded nucleic acids refers to a fluorescent dye that can insert between the planar bases of a double stranded nucleic acid, which makes the dye fluorescence much more intensively the dye in free form. After intercalated with the double-stranded nucleic acids, the intercalating dye 1 emits fluorescence with specific wavelength when it is excited. In the embodiment, the intercalating dye 1 may be, for example, fluorescent dye SYBR Green I. In other embodiments, the intercalating dye 1 may be selected from the group consisting of: SYBR Green I, SYBR Gold, ethidium bromide, LC Green, and EvaGreen.

The nucleic acid probe 2 includes a nucleic acid chain 21, a fluorescent reporter 22, and a fluorescent quencher 23. In the embodiment, the nucleic acid chain 21 is a peptide nucleic acid (PNA) chain, a locked nucleic acid (LNA) chain, a ribonucleic acid chain, or a deoxyribonucleic acid chain. The nucleic acid chain 21 has a length ranging from 15 mers to 70 mers. The fluorescent reporter 22 is conjugated to a first end 211 of the nucleic acid chain 21, and the fluorescent quencher 23 is conjugated to a second end 212 of the nucleic acid chain 21 opposing to the first end 211. The first end 211 of the nucleic acid chain 21 is the 5′ end or the 3′ end of the nucleic acid chain 21, and may be adjusted according to the actual requirement of the design of the nucleic acid probe 2, and the invention is not limited thereto. Those ordinarily skilled in the art can deduce that the second end 212 of the nucleic acid chain 21 is opposing to the first end 211 corresponding to the 5′ end or the 3′ end. In other words, the invention does not limit the fact that the fluorescent reporter 22 and the fluorescent quencher 23 are conjugated to the 5′ end or the 3′ end. It has been experimentally demonstrated that the conjugation of the fluorescent reporter 22 and the fluorescent quencher 23 to 5′ end or 3′ end does not affect the efficacy achieved by the nucleic acid probe 2, as will be detailed in Experimental example 3. In the embodiment, the fluorescent reporter 22 is selected from the group consisting of: HEX (Hexachlor-fluorescein, available from Thermo Fisher Scientific Inc.), Cy5 (Cyanine 5), Rhodamine X (ROX), Borondipyrromethene (Bodipy 630/650), and LightCycler® Red 640 (LCRed 640, available from Roche). In the embodiment, the fluorescent quencher 23 may be a black hole quencher (BHQ), for example, BHQ1, BHQ2, or BHQ3. In other embodiments, the fluorescent quencher 23 is selected from the group consisting of: DABCYL, the BHQ family (e.g., BHQ-1, BHQ-2, or BHQ-3, available from Sigma-Aldrich), Iowa Black (e.g., Iowa Black® FQ or Iowa Black® RQ, available from Integrated DNA Technologies, Inc.), the QSY family (e.g., QSY® 7, QSY® 9, QSY® 21, or QSY® 35, available from Thermo Fisher Scientific Inc.), and carboxytetramethyl rhodamine (referred to as TAM or TAMRA, available from Sigma-Aldrich).

In step S2, the nucleic acid probe 2 binds to the target nucleic acid fragment N_{T} to form a partial double stranded structure. The target nucleic acid fragment N_{T} is a nucleic acid fragment to be detected by the method according to the embodiment, and the nucleic acid sequence of the nucleic acid chain 21 of the nucleic acid probe 2 is designed to be complementary to a nucleic acid sequence of the target nucleic acid fragment N_{T}. Thus, the nucleic acid probe 2 binds to the target nucleic acid fragment N_{T} in a suitable environment (e.g., with a suitable buffer and at a suitable temperature) to form the partial double stranded structure.

After the nucleic acid probe 2 forms the partial double stranded structure with the target nucleic acid fragment N_{T}, step S3 is performed. That is, the intercalating dye 1 is intercalated in the partial double stranded structure. Next, the intercalating dye 1 is excited by an excitation light LEx with specific wavelength, so that the intercalating dye 1 generates another light with specific wavelength. Since the intercalating dye 1 is close to the fluorescent reporter 22, the fluorescent reporter 22 is excited by the energy of the light generated by the intercalating dye 1 during the process of fluorescence resonance energy transfer (FRET) (the hollow arrow as shown in FIG. 2A), so that the fluorescent reporter 22 emits a fluorescence signal L_{Em} with specific wavelength.

The embodiment is further illustrated by the fluorescent dye SYBR Green I as the intercalating dye 1. After the nucleic acid probe 2 forms the partial double stranded structure with the target nucleic acid fragment N_{T}, the fluorescent dye SYBR Green I is intercalated in the partial double stranded structure. Next, the fluorescent dye SYBR Green I is excited by an excitation light with wavelength of about 483 nm, so that the fluorescent dye SYBR Green I generates fluorescence with wavelength of about 522 nm. The fluorescent reporter 22 is excited by the energy of the fluorescence during the process of FRET, so that the fluorescent reporter 22 emits a fluorescence signal L_{Em} with specific wavelength.

In step S4, the target nucleic acid fragment N_{T} can be detected according to the fluorescence signal L_{Em}. Since the nucleic acid probe 2 is specifically bound to the target nucleic acid fragment N_{T}, the fluorescence intensity of the fluorescence signal L_{Em} with specific wavelength is proportional to the amount of the target nucleic acid fragment N_{T}, and can therefore be used to quantify the target nucleic acid fragment N_{T}.

A fluorescent quencher is a substance that can absorb the emission energy of a fluorophore (e.g., the fluorescent reporter of the invention) and dissipates the energy it absorbs in the form of thermal energy or by re-emitting light energy. Therefore, if the distance between the fluorescent quencher and the fluorescent reporter is close enough, the emission light of the fluorescent reporter can be absorbed by the nearby fluorescent quencher without being detected when the fluorescent reporter is excited. For example, about 94% of the quenching efficiency can be achieved when the fluorescent reporter is about 34 Å (angstrom) away from the fluorescent quencher. About 69% of the quenching efficiency can be achieved when the fluorescent reporter is about 48 Å away from the fluorescent quencher. If the fluorescent quencher absorbs the emission energy of the fluorophore and dissipates the energy it absorbs in the form of thermal energy, it is a dark quencher, such as DABCYL, BHQ (BHQ1, BHQ2 or BHQ3), Iowa Black, or QSY. However, after the fluorescent quencher in general absorbs the emission energy of the fluorophore, it may emit the energy it absorbs in the form of light. In some embodiments, the selection of the fluorescent reporter and the fluorescent quencher is considered based on the coordination of the emission wavelength of the emission light (fluorescence signal) of the fluorescent reporter and wavelength absorption range of the fluorescent quencher, and the emission wavelength of the emission light of the fluorescent quencher is greater than that of the fluorescence signal of the fluorescent reporter. For example, the fluorescent quencher is carboxytetramethyl rhodamine.

As shown in FIG. 2A, when the nucleic acid probe 2 is bound to the target nucleic acid fragment N_{T}, the distance between the fluorescent reporter 22 and the fluorescent quencher 23 on the nucleic acid probe 2 increases due to the base pairing. The distance makes the fluorescent quencher 23 not affect the fluorescence signal L_{Em} of the fluorescent reporter 22.

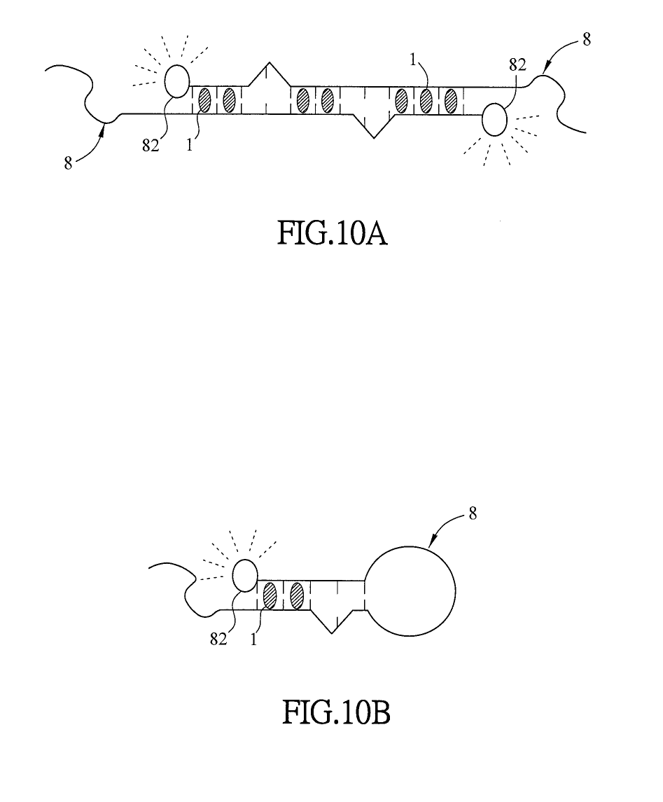

However, the nucleic acid probe would form intermolecular or intramolecular partial double-stranded structures, referred to as non-specific matches. FIGS. 2B and 2C are schematic diagrams of the nucleic acid probe 2 forming a non-specific match. FIGS. 10A and 10B are schematic diagrams showing the formation of a non-specific match of the nucleic acid probe that does not have a fluorescent quencher. Referring to FIG. 2B and FIG. 10A, when the nucleic acid sequences on the nucleic acid chain 21 of the nucleic acid probe 2 are complementary to form a non-specific match as shown in FIG. 2B (that is, when two nucleic acid probes 2 bind to each other), the intercalating dye 1 is intercalated in the partial double stranded structure because the nucleic acid probe 2 still forms partial double-stranded structures. As described above, when the intercalating dye 1 is excited by a light (excitation light) with specific wavelength, the fluorescent reporter 22 can be excited during the process of FRET, so that the fluorescent reporter 22 emits another fluorescence signal with specific wavelength. However, the fluorescent quencher 23 absorbs the fluorescence signal with specific wavelength emitted by the fluorescent reporter 22 due to the fluorescent reporter 22 being close to the fluorescent quencher 23. When detecting the fluorescence signal with specific wavelength emitted by the fluorescent reporter 22, only the fluorescence signal generated by the situation where the nucleic acid probe 2 forms a specific match with the target nucleic acid fragment N_{T} can be detected.

Similarly, as shown in FIG. 10A, when two nucleic acid probes 8 having no fluorescent quenchers bind to each other to form a non-specific match, the intercalating dye 1 is also intercalated in the double stranded structure formed by the two nucleic acid probes 8. When the intercalating dye 1 is excited by a light (excitation light) with specific wavelength, the fluorescent reporter 82 can also be excited during the process of FRET, so that the fluorescent reporter 82 emits another fluorescence signal with specific wavelength. Since the nucleic acid probe 8 does not have fluorescent quenchers, the fluorescence signal with specific wavelength generated by the fluorescent reporter 82 is not absorbed in the situation of the non-specific match to form a background signal. Thus, when detecting the fluorescence signal with specific wavelength emitted by the fluorescent reporter 82, not only the fluorescence signal generated by the situation where the nucleic acid probe 8 forms a specific match with the target nucleic acid fragment N_{T} can be detected, but also does the fluorescence signal generated by the situation where the two nucleic acid probes 8 form a non-specific match be detected. That is, the background signal generated from the non-specific match cannot be excluded. In contrast, the background signal generated from the non-specific match can be effectively eliminated using the nucleic acid probe 2 having the fluorescent quencher 23 according to the embodiment.

In addition to the intermolecular non-specific match formed by the binding of the two nucleic acid probes as described above, each nucleic acid probe per se may also form an intramolecular non-specific match. That is, each nucleic acid probe per se may form a partial double-stranded structure due to the base pairing. As shown in FIG. 2C, the fluorescent quencher 23 absorbs the fluorescence signal with specific wavelength emitted by the fluorescent reporter 22 due to the fluorescent reporter 22 being close to the fluorescent quencher 23. As shown in FIG. 10B, since the nucleic acid probe 8 does not have fluorescent quenchers, the fluorescence signal with specific wavelength generated by the fluorescent reporter 82 is not absorbed to form a background signal. In the embodiment, when the nucleic acid probe 2 is not bound to the target nucleic acid fragment N_{T} (that is, the nucleic acid probe 2 forms intermolecular or intramolecular partial double-stranded structures), the fluorescent quencher 23 absorbs the fluorescence signal emitted by the fluorescent reporter 22. The function of the intercalating dye 1, the fluorescent reporter 22, 82 and the fluorescent quencher 23 can be referred to the intermolecular non-specific match as described above, so the detailed descriptions thereof will be omitted.

A kit for detecting a target nucleic acid fragment is also provided. The kit comprises an intercalating dye which is configured to bind to double-stranded nucleic acids, and a nucleic acid probe. The nucleic acid probe includes a nucleic acid chain, a fluorescent reporter, and a fluorescent quencher. The fluorescent reporter is conjugated to a first end of the nucleic acid chain. The fluorescent quencher is conjugated to a second end of the nucleic acid chain opposing to the first end. The intercalating dye intercalates in a partial double stranded structure formed after the nucleic acid probe binds to the target nucleic acid fragment, and the target nucleic acid fragment is detected according to a fluorescence signal emitted by the fluorescent reporter which is excited through the intercalating dye.

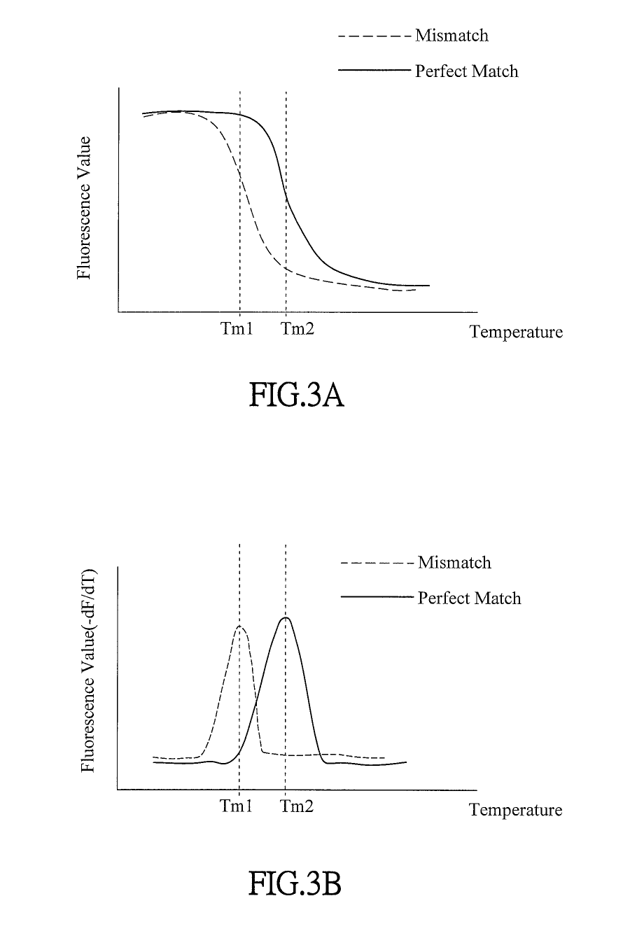

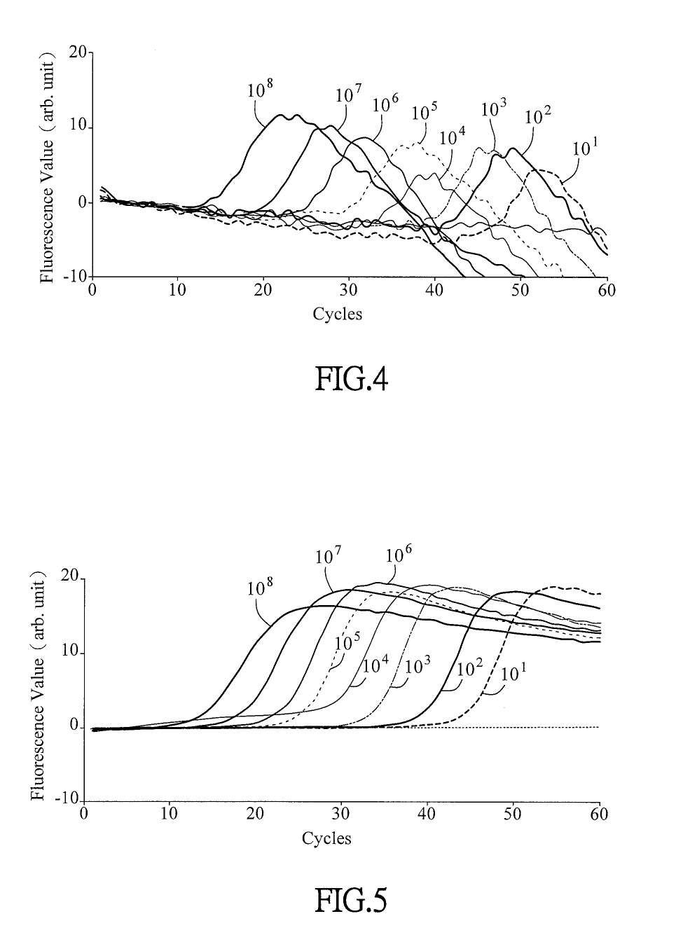

The method and the kit of the invention can be used for genotyping. Referring to FIGS. 3A and 3B, FIG. 3A is a schematic diagram of a melting curve showing that nucleic acid probes are perfectly matched or mismatched with target nucleic acid fragments, and FIG. 3B is a schematic diagram of a melting curve obtained by differentiating the melting curve of FIG. 3A. After the differential, the temperature corresponding to the peak of the melting curve is the melting temperature (Tm) of the nucleic acid probe and the target nucleic acid fragment. The melting temperature is the temperature at which the DNA double helix is unwound to two single strands. Thus, when the target nucleic acid fragment is completely complementary to the nucleic acid probe sequence to form a perfect match, more energy is required to allow the double helix to be unwound, so that the melting temperature Tm2 is higher. Conversely, when there is a mismatch between the target nucleic acid fragment and the nucleic acid probe sequence, the melting temperature Tm1 is lower. Since the sequence of the nucleic acid probe is designed according to the users' requirements, the perfect match or the mismatch between the target nucleic acid fragment and the nucleic acid probe can be determined by comparing the melting temperatures of the target nucleic acid fragment and the nucleic acid probe. The genotype of the target nucleic acid fragment can be further determined.

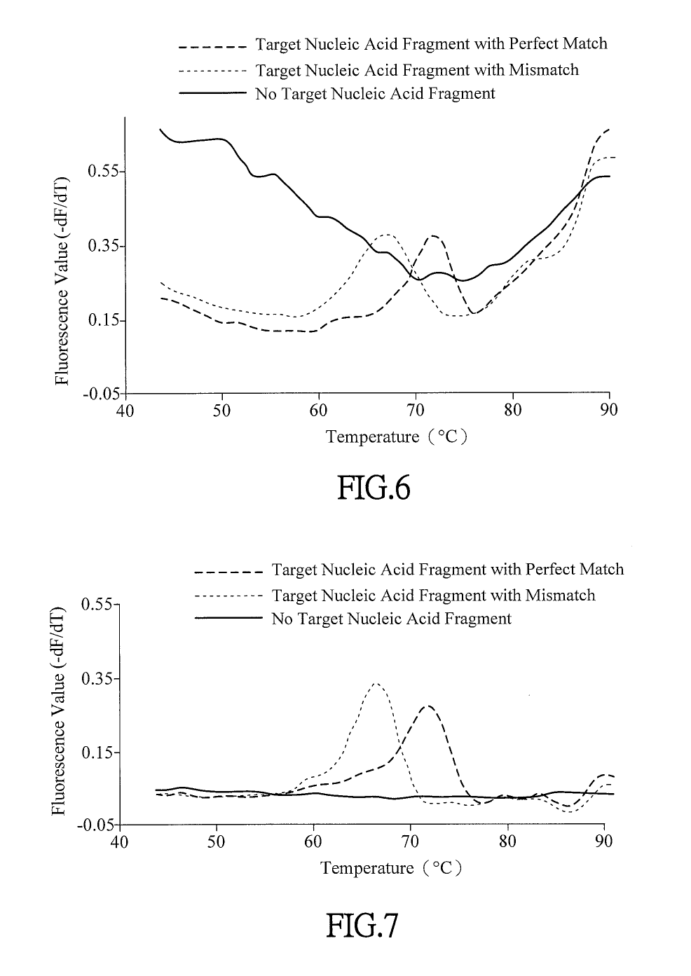

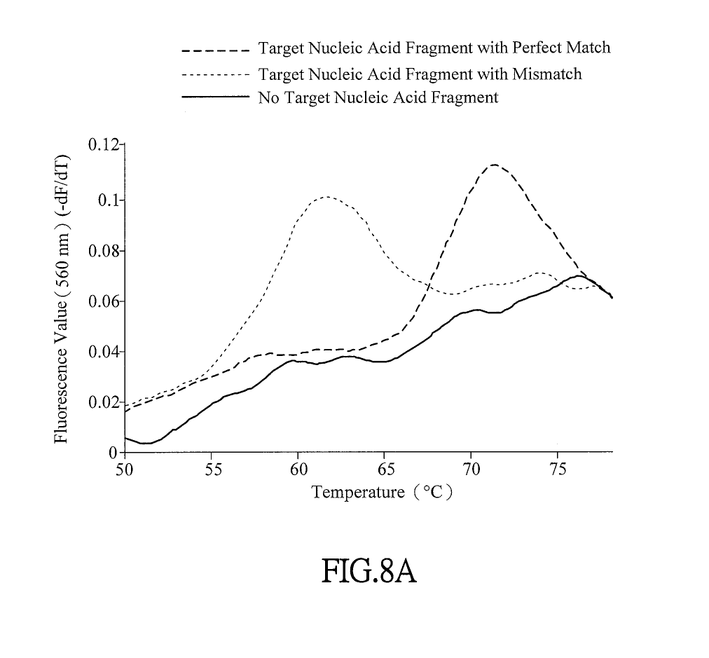

In addition, the method and the kit of the invention can provide an amplification curve with clearer signals to quantify the target nucleic acid fragment. Referring to FIG. 4 and FIG. 5, FIG. 4 is an amplification curve obtained using a nucleic acid probe that does not have a fluorescent quencher, and FIG. 5 is an amplification curve obtained using a nucleic acid probe having a fluorescent quencher. The number next to each amplification curve is the copy number of the target nucleic acid fragment as a template in the polymerase chain reaction, and the baseline is the line from the control group without the template. As can be seen from the figures, the signal of the amplification curve obtained using the nucleic acid probe without fluorescent quenchers is chaotic and difficult to interpret. Conversely, the signal of the amplification curve obtained using the nucleic acid probe having fluorescent quenchers is clearer and easy to interpret. Quantification of the target nucleic acid fragment according to the amplification curve is the general knowledge in the art to which the invention pertains, so the detailed descriptions thereof will be omitted.

In the below descriptions, Experimental examples illustrate the method and the kit of the invention can be used for genotyping with the analysis of the melting curve.

### Experimental Example 1: Genotyping was Performed Using a Melting Curve

In Experimental example 1, the nucleic acid probe was used to detect the point mutations (r. 2063 A>G or r. 2064 A>G) on domain V of 23s rRNA gene of Mycoplasma pneumoniae. First, the gene fragments (target nucleic acid fragments) of M. pneumoniae to be detected were amplified using PCR. Further, by using the analysis of the melting curve, the existence of the point mutations on the gene fragments (target nucleic acid fragments) was distinguished by the peak position in the melting curve.

The polymerase chain reaction was carried out with the following reactants: 20 μl of reaction volume was added with 1×PCR mixture (PCR Mix) containing 50 mM Tris (pH 8.5), 3 mM magnesium chloride (MgCl₂), 0.5 mg/ml BSA and dNTP (200 μM each); 0.25 μM primer MP-F: 5′-TCCAGGTACGGGTGAAGACA-3′; 0.083 μM primer MP-R: 5′-GCTCCTACCTATTCTCTACATGAT-3′; 0.25 μM nucleic acid probe: 5′-ROX-GCGCAACGGGACGGAAAGAC-BHQ1-3′; 0.5 U Taq DNA polymerase; 1/20000×SYBR Green I; and the nucleic acid sample of M. pneumoniae (target nucleic acid fragments). There is considerable effect regarding the concentration of the nucleic acid probe in the range of 0.12 μM to 1 μM.

The reaction conditions of PCR were as follows: the reaction temperature was 95° C. and the reaction was performed for 5 minutes, repeated 70 cycles at 95° C. for 5 seconds, 56° C. for 3 seconds, 72° C. for 15 seconds, and each cycle at 56° C. to detect fluorescence signals one time. After 70 cycles, the reaction was performed at 72° C. for 1 minute. Finally, the melting curve was analyzed using the following conditions: after performing the reaction at 95° C. for 30 seconds, the temperature was reduced to 40° C. and the reaction was performed for 10 seconds, followed by increasing the temperature to 95° C. at an increasing rate of 0.7° C. per second. The fluorescence signal was detected at 610 nm with a detection rate of one time per second during the process.

When the fluorescence signal was detected, the wavelength of the excitation light was set at about 483 nm, and the wavelength of the detection light was set similarly to the maximum emission light of the fluorescent reporter of the nucleic acid probe. In Experimental example 1, the fluorescent reporter was ROX, so that the wavelength of the detection light was set at 610 nm. If LCRed 640 was used as the fluorescent reporter, the wavelength of the detection light was set at 640 nm.

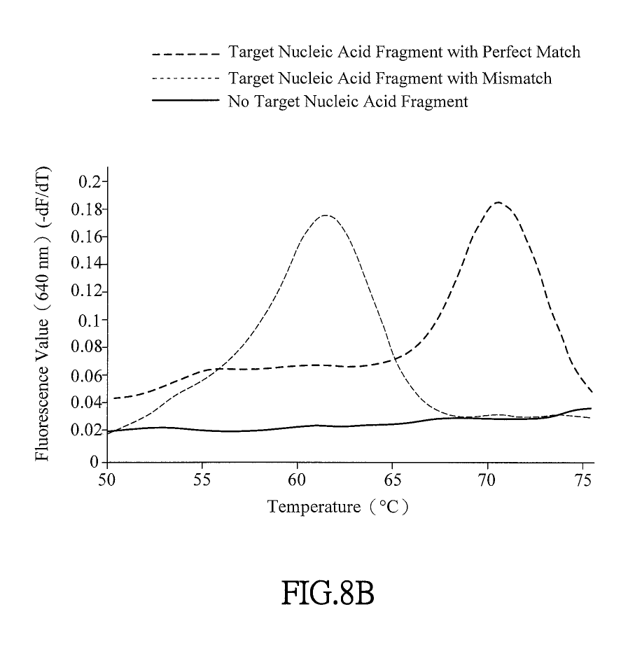

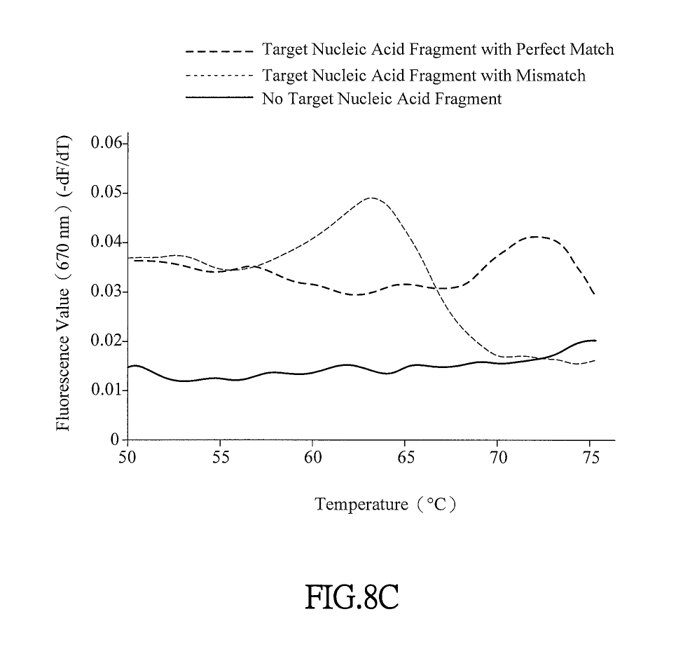

The curve of the fluorescence value (F) versus temperature (T) of the fluorescence signal with 610 nm detected with a detection rate of one time per second was plotted, and an initial melting curve was obtained. The melting curve was obtained using the color compensation formula to deduct the background value of SYBR Green I. Afterward, the melting curve was differentiated from temperature, and the negative value (−dF/dT) was taken, such that a differentiated melting curve was obtained (FIG. 7). The aforesaid experiment was also performed using the nucleic acid probe having no fluorescent quenchers, and the experimental results are shown in FIG. 6.

In addition, the fluorescence signal detected at 56° C. (annealing stage) per cycle was also plotted to obtain an initial amplification curve. The amplification curve was obtained using the color compensation formula to deduct the background value of SYBR Green I (figures not shown). The target nucleic acid fragments can be further quantified using the amplification curve.

Referring to FIG. 6 and FIG. 7, FIG. 6 is a melting curve obtained using a nucleic acid probe that does not have a fluorescent quencher in Experimental example 1, and FIG. 7 is a melting curve obtained using a nucleic acid probe having a fluorescent quencher in Experimental example 1. As shown in FIG. 6, in the absence of the target nucleic acid fragments, the nucleic acid probe per se produces a strong background signal. The melting curves regarding the target nucleic acid fragments with perfect matches and the target nucleic acid fragments with mismatches were affected by the background signal, and the interpretation of the peak position was interfered. However, as shown in FIG. 7, there is almost no background signal when using the nucleic acid probe having fluorescent quenchers in the absence of the target nucleic acid fragments. The melting curve is clearer and easy to interpret because the background signal is effectively reduced.

Referring to FIG. 7, the peak position of the melting curve at 72° C. is the wild type target nucleic acid fragment, and the peak position of the melting curve at 68° C. is the target nucleic acid fragment with point mutations. Since the nucleic acid sequence of the nucleic acid probe is designed according to the wild type target nucleic acid fragment, there is a perfect match between the nucleic acid probe and the wild type target nucleic acid fragment, and there is a mismatch between the nucleic acid probe and the target nucleic acid fragment with point mutations. That is, the wild type target nucleic acid fragment is the target nucleic acid fragment with perfect matches, and the target nucleic acid fragment with point mutations is the target nucleic acid fragment with mismatches. Thus, the melting temperature of the nucleic acid probe and the wild type target nucleic acid fragment is higher than that of the target nucleic acid fragment with point mutations. When there is no target nucleic acid fragment (gene fragment of M. pneumoniae), there is no peak in the melting curve.

### Experimental Example 2: Comparison of Different Fluorescent Reporters and Fluorescent Quenchers

In Experimental example 2, the nucleic acid probe was used to detect the gene mutation on codon 12 of KRAS gene in human cells, and the genomic DNA of K562 cell line was used as a wild type DNA template (i.e., the target nucleic acid fragment with perfect matches). The genomic DNA of TSGH cell line which has a single base mutation on codon 12 of KRAS was used as a mutant DNA template (i.e., the target nucleic acid fragment with mismatches).

In Experimental example 2, in order to compare the effects of different fluorescent reporters and fluorescent quenchers, 5′ end and 3′ end of the nucleic acid probe were respectively labeled with different fluorescent reporters and fluorescent quenchers, and different nucleic acid probes were subjected to PCR. The primers and the nucleic acid probes used in Experimental example 2 are summarized in Table 1, wherein the sequences of the primers and the nucleic acid probes are represented by 5′ end to 3′ end.

TABLE 1

| | | 5′ end | 3′ end |
|---|---|---|---|
| Primer/Probe | Sequence (5′→3′) | labeling | labeling |
| Forward Primer | ATAAGGCCTGCTGAAAATGAC | | |
| | TG | | |
| Reverse Primer | CAAAGAATGGTCCTGCACCAG | | |
| Probe-5HEX-3Q1 | CCTACGCCACCAGCTCCAAC | 5′HEX | 3′BHQ1 |
| Probe-5R640-3Q2 | CCTACGCCACCAGCTCCAAC | 5′LCRed 640 | 3′BHQ2 |
| Probe-5Cy5-3Q3 | CCTACGCCACCAGCTCCAAC | 5′Cy5 | 3′BHQ3 |

The polymerase chain reaction was carried out with the reactants listed in Table 2, with a total reaction volume of 20 μl.

TABLE 2

| | | Volume | Final |
|---|---|---|---|
| Reactant | Concentration | (μl) | Concentration |
| KAPA buffer | 5X | 4 | 1X |
| 4 dNTPs | 10 mM | 0.4 | 200 μM each |
| Magnesium chloride | 125 mM | 0.4 | 2.5 mM |
| Forward Primer | 10 μM | 2 | 1 μM |
| Reverse Primer | 10/3 μM | 1 | 0.17 μM |
| Genomic DNA* (gDNA) | 5 ng/μl | 2 | 0.5 ng/μl |
| SYBR Green I | 10X | 1 | 0.5X |
| KAPA enzyme | 1 U/μl | 0.1 | 0.005 U/μl |
| Probe | 10 μM | 1 | 0.5 μM |
| Water | | 8.1 | |
| Total amount | | 20 | |

The reaction conditions of PCR were as follows: the reaction temperature was 95° C. and the reaction was performed for 5 minutes, repeated 50 cycles at 98° C. for 10 seconds, 60° C. for 10 seconds, 72° C. for 20 seconds, and each cycle at 60° C. to detect fluorescence signals one time. After 50 cycles, the reaction was performed at 72° C. for 2 minutes. Finally, the melting curve was analyzed using the following conditions: after performing the reaction at 95° C. for 1 second, the temperature was reduced to 40° C. and hold for 1 second, followed by increasing the temperature to 95° C. at an increasing rate of 0.06° C. per second. The fluorescence signal was detected with a detection rate of two times per ° C. during the process.

When the fluorescence signal was detected, the wavelength of the excitation light was set at about 483 nm, and the wavelength of the detection light was set similarly to the maximum emission light of the fluorescent reporter of the nucleic acid probe. In Experimental example 2, HEX, LCRed 640 and Cy5 were used as the fluorescent reporters, so that the wavelengths of the detection light were set at 560 nm, 640 nm and 670 nm, respectively.

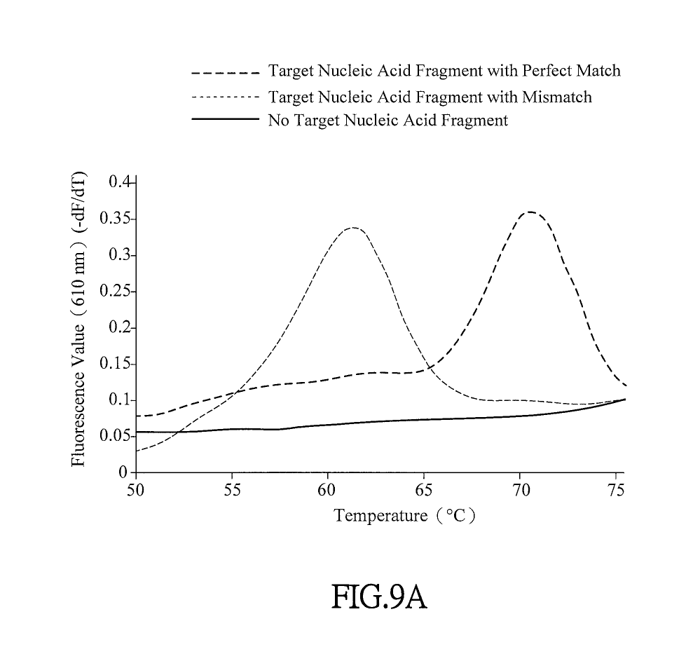

Subsequently, the melting curves were plotted according to the method as described in Experimental example 1, and shown in FIG. 8A, FIG. 8B and FIG. 8C, respectively. The nucleic acid probe used in FIG. 8A comprises HEX as a fluorescent reporter, and comprises BHQ1 as a fluorescent quencher. The nucleic acid probe used in FIG. 8B comprises LCRed 640 as a fluorescent reporter, and comprises BHQ2 as a fluorescent quencher. The nucleic acid probe used in FIG. 8C comprises Cy5 as a fluorescent reporter, and comprises BHQ3 as a fluorescent quencher.

Referring to FIGS. 8A, 8B and 8C, which are melting curves obtained using different fluorescent reporters and fluorescent quenchers in Experimental example 2 of the present disclosure. As shown in the figures, the peak positions of the melting curve regarding the target nucleic acid fragment with perfect matches are at 72° C., the peak positions of the melting curve regarding the target nucleic acid fragment with mismatches are at 62° C., and the background signal is very low in the absence of the target nucleic acid fragments. It is known that using fluorescent reporters such as HEX, LCRed 640 and Cy5 and fluorescent quenchers such as BHQ1, BHQ2 and BHQ3 can effectively eliminate the background signals generated by the non-specific match, so that the melting curve is clearer and easier to interpret for genotyping.

### Experimental Example 3: Comparison the Difference of Fluorescent Reporters at 5′ End or 3′ End of Nucleic Acid Probes

In Experimental example 3, the target nucleic acid fragments, the primers, the reactants for PCR, and the operation conditions of PCR are the same as those in Experimental example 2. However, Experimental example 3 explores whether the conjugation of the fluorescent reporter to 5′ end or 3′ end of the nucleic acid probe affects the efficacy of the nucleic acid probe, so the nucleic acid probes conjugated with fluorescent reporters at different ends were used for PCR, as listed in Table 4.

TABLE 4

| | | 5′ end | 3′ end |
|---|---|---|---|
| Probe | Sequence (5′→3′) | labeling | labeling |
| Probe-5ROX-3Q2 | CCTACGCCACCAGCTCCAAC | 5′ROX | 3′BHQ2 |
| Probe-5Q2-3ROX | CCTACGCCACCAGCTCCAAC | 5′BHQ2 | 3′ROX |

Similarly, when the fluorescence signal was detected, the wavelength of the excitation light was set at about 483 nm, and the wavelength of the detection light was set similarly to the maximum emission light of the fluorescent reporter of the nucleic acid probe. In Experimental example 3, the fluorescent reporter was ROX, so that the wavelength of the detection light was set at 610 nm.

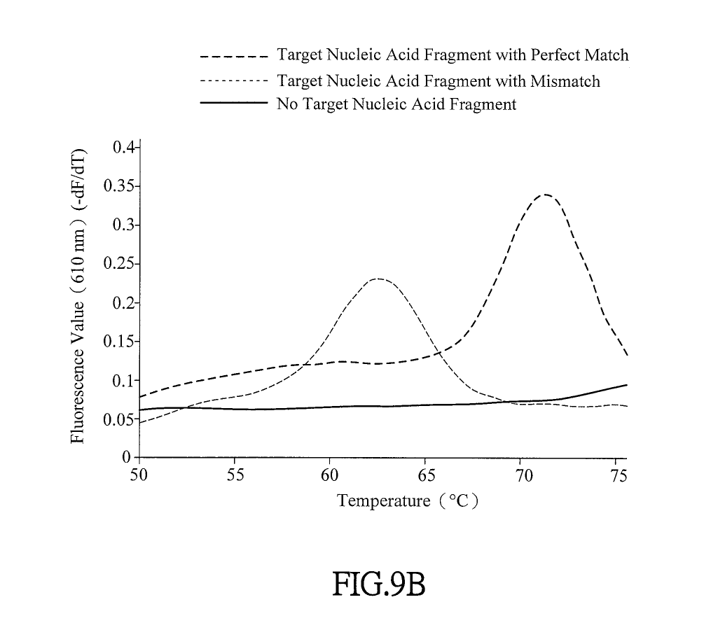

Subsequently, the melting curves were plotted according to the method as described in Experimental example 1, and the experimental results were shown in FIG. 9A and FIG. 9B, respectively. The nucleic acid probe used in FIG. 9A comprises ROX at 5′ end as a fluorescent reporter, and comprises BHQ2 at 3′ end as a fluorescent quencher. The nucleic acid probe used in FIG. 9B comprises BHQ2 at 5′ end as a fluorescent quencher, and comprises ROX at 3′ end as a fluorescent reporter.

Referring to FIG. 9A and FIG. 9B, the peak positions of the melting curve regarding the target nucleic acid fragment with perfect matches are at 72° C., the peak positions of the melting curve regarding the target nucleic acid fragment with mismatches are at 62° C., and the background signal is very low in the absence of the target nucleic acid fragments. It is known that conjugation of the fluorescent reporter and the fluorescent quencher to any end (5′ end or 3′ end) of the nucleic acid chain does not affect the efficacy of the nucleic acid probe of the invention. The nucleic acid probe can effectively eliminate the background signals generated by the non-specific match, so that the melting curve is clearer and easier to interpret for genotyping.

In summary, the method and the kit of the present disclosure are used to detect target nucleic acid fragments using the nucleic acid probe including the fluorescent reporter and the fluorescent quencher, and the intercalating dye. When the nucleic acid probe forms intramolecular or intermolecular partial double-stranded structures (i.e., a non-specific match occurs), the fluorescent quencher absorbs the fluorescence signal with specific wavelength emitted by the fluorescent reporter due to the fluorescent reporter being close to the fluorescent quencher. That is, when the nucleic acid probe is not bound to the target nucleic acid fragment, the fluorescent quencher absorbs the fluorescence signal emitted by the fluorescent reporter. Therefore, when using in the polymerase chain reaction, the background signal generated by the non-specific match can be effectively eliminated, so that the amplification curve or the melting curve is clearer and easier to interpret to efficiently quantify the target nucleic acid fragments or to perform genotyping.

Although the present invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the present invention.

What is claimed is:

1. A method for detecting a target nucleic acid fragment, the method comprising the following steps:
preparing a nucleic acid probe and an intercalating dye which is configured to bind to double-stranded nucleic acids, wherein the nucleic acid probe includes a nucleic acid chain, a fluorescent reporter, and a fluorescent quencher, the fluorescent reporter is conjugated to a first end of the nucleic acid chain, and the fluorescent quencher is conjugated to a second end of the nucleic acid chain opposing to the first end;
binding the nucleic acid probe to the target nucleic acid fragment to form a partial double stranded structure;
intercalating the intercalating dye in the partial double stranded structure, so that, after the intercalating dye is excited by an irradiation, the fluorescent reporter is excited through the intercalating dye to emit a fluorescence signal; and
detecting the target nucleic acid fragment according to the fluorescence signal.

2. The method of claim 1, wherein when the nucleic acid probe is not bound to the target nucleic acid fragment, the fluorescent quencher absorbs the fluorescence signal emitted by the fluorescent reporter.

3. The method of claim 1, wherein the nucleic acid chain is a peptide nucleic acid chain, a locked nucleic acid chain, a ribonucleic acid chain, or a deoxyribonucleic acid chain.

4. The method of claim 1, wherein the nucleic acid chain has a length ranging from 15 mers to 70 mers.

5. The method of claim 1, wherein the fluorescent reporter is selected from the group consisting of: HEX, Cy5, ROX, Bodipy 630/650, and LCRed 640.

6. The method of claim 1, wherein the fluorescent quencher is selected from the group consisting of: DABCYL, BHQ, Iowa Black, QSY, and carboxytetramethyl rhodamine.

7. The method of claim 1, wherein the intercalating dye is selected from the group consisting of: SYBR Green I, SYBR Gold, ethidium bromide, LC Green, and EvaGreen.

8. A kit for detecting a target nucleic acid fragment, comprising:
an intercalating dye which is configured to bind to double-stranded nucleic acids; and
a nucleic acid probe, wherein the nucleic acid probe includes:
a nucleic acid chain;
a fluorescent reporter conjugated to a first end of the nucleic acid chain; and
a fluorescent quencher conjugated to a second end of the nucleic acid chain opposing to the first end,
wherein the intercalating dye intercalates in a partial double stranded structure formed after the nucleic acid probe binds to the target nucleic acid fragment, and the target nucleic acid fragment is detected according to a fluorescence signal emitted by the fluorescent reporter which is excited through the intercalating dye.

9. The kit of claim 8, wherein when the nucleic acid probe is not bound to the target nucleic acid fragment, the fluorescent quencher absorbs the fluorescence signal emitted by the fluorescent reporter.

10. The kit of claim 8, wherein the nucleic acid chain is a peptide nucleic acid chain, a locked nucleic acid chain, a ribonucleic acid chain, or a deoxyribonucleic acid chain.

11. The kit of claim 8, wherein the nucleic acid chain has a length ranging from 15 mers to 70 mers.

12. The kit of claim 8, wherein the fluorescent reporter is selected from the group consisting of: HEX, Cy5, ROX, Bodipy 630/650, and LCRed 640.

13. The kit of claim 8, wherein the fluorescent quencher is selected from the group consisting of: DABCYL, BHQ, Iowa Black, QSY, and carboxytetramethyl rhodamine.

14. The kit of claim 8, wherein the intercalating dye is selected from the group consisting of: SYBR Green I, SYBR Gold, ethidium bromide, LC Green, and EvaGreen.
